(19) United States
(12) Patent Application Publication
(10) Pub. No.: US 2026/0151551 A1
(43) Pub. Date: Jun. 4, 2026

(54) CONNECTION UNIT FOR VACUUM-TIGHT FLUIDIC CONNECTION OF A VACUUM WOUND DRESSING TO A VACUUM SOURCE, VACUUM WOUND TREATMENT KIT AND VACUUM WOUND TREATMENT SYSTEM

(71) Applicant: PAUL HARTMANN AG, Heidenheim (DE)

(72) Inventor: Martin DEIBLER, Herbrechtingen (DE)

(73) Assignee: PAUL HARTMANN AG, Heidenheim (DE)

(21) Appl. No.: 19/140,238

(22) PCT Filed: Dec. 6, 2023
(86) PCT No.: PCT/EP2023/084525

§ 371 Date: Jun. 17, 2025

(30) Foreign Application Priority Data
Dec. 19, 2022 (DE) 10 2022 133 929.7

(51) Int. Cl. A61M 1/00

(52) U.S. Cl. CPC A61M 1/912; A61M 2206/20

(57) ABSTRACT

The invention relates to a connection unit (20) for vacuum-tight fluidic connection of a vacuum wound dressing (16) to a vacuum source (12), with an elongated connecting tube (22), having a distal end section (24) with a fluid inlet (26) and a proximal end section (28) wit a fluid outlet (30), wherein the fluid inlet can be fluidically connected to the vacuum wound dressing in a vacuum-tight manner, wherein the fluid outlet can be fluidically connected to the vacuum source in a vacuum-tight manner, and wherein a suction lumen (32) of the connecting tube extends from the fluid inlet to the fluid outlet, wherein according to the invention: the suction lumen comprises a fluid-guiding structure (46), which is designed to deflect a fluid flow flowing through the suction lumen multiple times before reaching the fluid outlet; the fluid-guiding structure comprises at least one fluid-guiding element (48) extending in the suction lumen for deflecting the fluid flow; and the fluid guiding element is formed by integrally connecting, in certain areas, a first wall section (50) of a tube wall (52) of the connecting tube to a second wall section (54) of the tube wall opposite the first wall section.

The present invention relates to a connection unit for vacuum-tight fluidic connection of a vacuum wound dressing to a vacuum source. The present invention also relates to a vacuum wound treatment kit comprising such a connection unit. In addition, the present invention relates to a vacuum wound treatment system comprising a vacuum source and such a vacuum wound treatment kit.

The vacuum treatment of wounds, also referred to as NPWT=Negative-Pressure Wound Therapy, is an innovative method of treating wounds with a wide range of indications. These include, for example, acute skin and soft tissue defects, wound healing disorders, chronic wounds etc. The aim of vacuum treatment is to stimulate the growth of granulation tissue and to promote the healing process. In the context of vacuum treatment, this aim is pursued by producing a vacuum in the region of a wound, thereby enabling wound exudate to be drained effectively from the wound. Drained wound exudate is absorbed by a vacuum wound dressing applied to the wound.

Vacuum wound dressings used in vacuum treatment typically comprise an air-impermeable covering layer for airtight sealing of the wound. A connection opening for vacuum-tight fluidic connection of the wound space to a vacuum source is formed in the covering layer. When the vacuum wound dressing is applied to a wound as intended and the vacuum source has been fluidically connected to the connection opening in a vacuum-tight manner, a vacuum can be produced in the wound space by the vacuum source. To fluidically connect the vacuum wound dressing to the vacuum source in a vacuum-tight manner, a connection unit comprising an elongate single-or multi-lumen connecting tube is typically used.

A vacuum wound treatment kit comprising a connection unit of the type in question is known from WO 2016 184 916 A1, for example. A filter unit is arranged at the distal end section of the connecting tube of the connection unit. The filter unit is assigned to the fluid inlet of the distal end section and prevents wound exudate from entering the suction lumen of the connecting tube.

WO 2011 135 287 A1 describes a vacuum wound treatment kit comprising another connection unit of the type in question. In this case, the connection opening of the associated vacuum wound dressing is assigned a filter unit which prevents wound exudate escaping from the vacuum wound dressing.

In principle, a situation where wound exudate gets into the vacuum source should be avoided in vacuum treatment. This could contaminate and/or damage the vacuum source. In the vacuum wound treatment kits which are described in the abovementioned documents, this is ensured by filter units. However, it can happen that the filter units are wetted by wound exudate and consequently become clogged before the absorption capacity of the vacuum wound dressing is reached. This is the case precisely when wound exudate is carried in a surge or surges in the direction of the connection unit. It may then be necessary to replace the vacuum wound dressing prematurely.

It is the underlying object of the invention to provide a connection unit which, when used as intended in vacuum treatment, enables the most efficient use possible of the absorption capacity of the vacuum wound dressing used.

According to the invention, this object is achieved by a connection unit as claimed in claim 1, by a vacuum wound treatment kit as claimed in claim 19, and by a vacuum wound treatment system as claimed in claim 20.

The dependent claims and the description indicate advantageous variants and embodiments.

According to the invention, therefore, a connection unit for vacuum-tight fluidic connection of a vacuum wound dressing to a vacuum source is provided. The connection unit comprises an elongate single-or multi-lumen connecting tube. The connecting tube comprises a distal end section with a fluid inlet and a proximal end section with a fluid outlet. The fluid inlet can be fluidically connected to the vacuum wound dressing in a vacuum-tight manner. The fluid outlet can be fluidically connected to the vacuum source in a vacuum-tight manner. A suction lumen of the connecting tube extends from the fluid inlet to the fluid outlet. When the connection unit is used as intended in vacuum treatment, a vacuum is communicated from the vacuum source to the wound space via the suction lumen.

Here, a “vacuum-tight fluidic connection” should be understood to mean that, taking into account the vacuum source used, the vacuum necessary for the vacuum treatment of wounds can be maintained in the fluidically interconnected cavities. In the context of vacuum treatment, a pressure difference between the air pressure within the vacuum wound dressing and the ambient air pressure that is at least 20 mm Hg (millimeters of mercury) to a maximum of 250 mm Hg is typically set. 1 mm Hg corresponds to one torr or 133.322 Pa (pascals).

The terms “distal” and “proximal” describe the arrangement relative to the vacuum source. A proximal section of an element is situated closer to the vacuum source than a distal section of the same element. Thus, the proximal end section of the connecting tube is closer to the vacuum source than the distal end section, for example, when the connecting tube is used as intended.

It is now envisaged that the suction lumen comprises a fluid-guiding structure, which is designed to deflect a fluid flow flowing through the suction lumen multiple times before it reaches the fluid outlet, that, to deflect the fluid flow, the fluid-guiding structure comprises at least one fluid-guiding element extending in the suction lumen, and that the fluid-guiding element is formed by material joining, in some region or regions, of a first wall section of a tube wall of the connecting tube to a second wall section of the tube wall opposite the first wall section.

The fluid-guiding structure according to the invention is an effective means of avoiding wound exudate that surges into the connecting tube from reaching the proximal end section. If wound exudate surges into the suction lumen, the fluid flow formed by the wound exudate is deflected multiple times by the fluid-guiding structure. The multiple deflection leads to the surging fluid flow being interrupted. It is only when the absorption capacity of the vacuum wound dressing for wound exudate is actually reached and, as a consequence, the connecting tube is continuously filled with wound exudate that the wound exudate gets into the proximal end section. Because wound exudate surging into the suction lumen does not reach the proximal end section and thus the vacuum source, it is possible to dispense with a filter unit on the distal end section of the connecting tube and on the covering layer of the vacuum wound dressing. If a filter unit arranged in this way is dispensed with, premature clogging of the filter unit also does not take place.

To deflect the fluid flow, the fluid-guiding structure comprises at least one fluid-guiding element extending in the suction lumen. The fluid-guiding structure preferably comprises a plurality of fluid-guiding elements extending in the suction lumen. The fluid-guiding element or at least one of the fluid-guiding elements preferably has a straight profile. However, the fluid-guiding element or at least one of the fluid-guiding elements may also have a bent profile.

By materially joining the first wall section to the second wall section in some region or regions, it is possible to create a fluid-guiding element or a plurality of fluid-guiding elements with little effort in terms of construction. The material joint between the first wall section and the second wall section is preferably embodied as a welded joint or as an adhesive joint. At least one of the wall sections for the formation of the fluid-guiding element preferably comprises an indentation which projects in the direction of the other wall section. Because the first wall section is materially joined to the second wall section only in some region or regions, the fluid-guiding element also extends only in a limited region of the suction lumen.

According to one preferred embodiment, the connection unit is free from a filter unit.

According to one alternative embodiment, an air-permeable and liquid-impermeable filter unit is preferably arranged in the region of the proximal end section. Because the filter unit is arranged in the proximal end section, premature clogging of the filter unit is avoided in an effective manner by means of the fluid-guiding structure according to the invention. As a particular preference, the filter unit is arranged on a connection element having an inlet and an outlet, wherein a connection-element inlet section comprising the inlet projects through the fluid outlet of the proximal end section into the suction lumen, and wherein the outlet of the connection element is arranged outside the suction lumen and can be fluidically connected to a vacuum source in a vacuum-tight manner. The fluid outlet of the proximal end section can then be fluidically connected to the vacuum source in a vacuum-tight manner by means of the connection element.

The connecting tube can be of single-lumen or multi-lumen design. In the case of a single-lumen connecting tube, there is only a single tube lumen, namely the suction lumen. In the case of a multi-lumen connecting tube, the tube lumen is divided into a number of partial lumens, which each extend from the distal end section to the proximal end section. The partial lumens are preferably connected in parallel with one another to the fluid inlet of the distal end section, wherein each of the partial lumens is assigned a respectively dedicated passage in the region of the proximal end section. In the case of such a multi-lumen connecting tube, a first one of the partial lumens serves as the suction lumen when the connection unit is used as intended. The passage assigned to this partial lumen forms the fluid outlet of the proximal end section. A second one of the partial lumens can be used as a purging lumen and/or as a measurement lumen, for example.

According to one preferred embodiment, it is envisaged that the material joint between the first wall section and the second wall section is in the form of a seam. A material joint in the form of a seam can be formed with little effort. For example, the first wall section and the second wall section are welded to one another to form the fluid-guiding element, with the result that the material joint in the form of a seam is a weld seam. In comparison with a spot-type connection, for example, a connection in the form of a seam furthermore has the advantage that an elongate fluid-guiding element can be obtained. Particularly precise deflection of the fluid flow is thereby made possible.

According to one preferred embodiment, it is envisaged that the fluid-guiding structure comprises at least two fluid-guiding elements. By increasing the number of the fluid-guiding elements, it is possible to achieve particularly precise multiple deflection of the fluid flow in the suction lumen. The fluid-guiding structure preferably comprises at least three fluid-guiding elements, preferably at least four fluid-guiding elements. However, increasing the number of fluid-guiding elements increases the effort as regards the manufacture of the connection unit. Moreover, the free volume of the suction lumen is reduced. Accordingly, limitation of the number of fluid-guiding elements is also advantageous. As a particular preference, the fluid-guiding structure comprises at least two and at most twenty fluid-guiding elements.

According to one preferred embodiment, it is envisaged that the fluid-guiding structure is designed to block any straight-line connection between an imaginary first plane, which is arranged in the distal end section and is aligned orthogonally with respect to the longitudinal extent of the connecting tube, and an imaginary second plane, which is arranged in the proximal end section and is aligned orthogonally with respect to the longitudinal extent of the connecting tube. It is assumed that the above feature is achieved when each straight-line connection between the two planes is blocked when the connecting tube is stretched straight. Blocking each straight-line connection is a particularly effective way of avoiding the surging transfer of wound exudate from the distal end section to the proximal end section.

According to one preferred embodiment, it is envisaged that the fluid-guiding structure is designed to deflect the fluid flow at least once, preferably multiple times, through an angle which is at least 90°. Deflection through such a large angle effectively interrupts surging fluid flow, and therefore the surging fluid flow as such cannot get into the proximal end section. The fluid-guiding structure is preferably designed to deflect the fluid flow at least once, preferably multiple times, through an angle which is at least 120°, particularly preferably at least 150°.

The flow direction of the fluid flow in a first section of the suction lumen is preferably opposite to the flow direction of the fluid flow in a second section of the suction lumen. The fluid flow is thus deflected at least once through an angle which is 180°. A surging fluid flow is thereby interrupted in a particularly effective manner. Moreover, sections with opposite flow directions lengthen the flow path in the suction lumen. It is thereby possible to assess in a particularly precise way whether the vacuum wound treatment kit should or should not be replaced.

According to one preferred embodiment, it is envisaged that the fluid-guiding structure defines a meandering or labyrinthine fluid passage in the suction lumen. In the case of such a fluid passage, the flow cross section is significantly reduced as compared with the flow cross section of the same connecting tube without a fluid-guiding structure. The reduction in the flow cross section is associated with an increase in the flow resistance and a reduction in the flow rate. The reduction in the flow rate has the advantage that wound exudate flowing through the suction lumen can be analyzed more easily. In particular, a sensor unit for analyzing the wound exudate is arranged in the suction lumen. However, the wound exudate can also be analyzed, in particular visually, from outside the connecting tube.

According to one preferred embodiment, it is envisaged that the fluid-guiding element or at least one of the fluid-guiding elements extends transversely to the longitudinal extent of the connecting tube, at least in some section or sections. A section of a fluid-guiding element which extends in this way or a fluid-guiding element which extends in this way can effectively deflect a fluid flow flowing in the longitudinal extent of the connecting tube. There is preferably a plurality of fluid-guiding elements which extend transversely to the longitudinal extent of the connecting tube and which are arranged offset relative to one another and in series in the longitudinal extent of the connecting tube. By means of such an arrangement of fluid-guiding elements, a meandering or labyrinthine fluid passage can be defined in the suction lumen.

According to one preferred embodiment, it is envisaged that the fluid-guiding element or at least one of the fluid-guiding elements extends in the longitudinal extent of the connecting tube, at least in some section or sections. A section of a fluid-guiding element which extends in this way or a fluid-guiding element which extends in this way divides the suction lumen into a plurality of sections that are arranged side by side and are extended in the longitudinal extent of the connecting tube. With such a division of the suction lumen, it is advantageously possible to ensure that the flow direction in one section is opposite to the flow direction in the other section.

According to one preferred embodiment, it is envisaged that the fluid-guiding element or at least one of the fluid-guiding elements is formed by direct material joining of the first wall section to the second wall section. Such a design of a fluid-guiding element is easy to implement in terms of manufacturing technology. The first wall section and the second wall section are preferably joined directly to one another materially by welding. However, the first wall section and the second wall section can also be joined directly to one another materially by adhesive bonding.

According to one preferred embodiment, it is envisaged that the fluid-guiding element or at least one of the fluid-guiding elements is formed by indirect material joining of the first wall section to the second wall section. By means of indirect material joining, it is also possible to obtain a suitable fluid-guiding element or suitable fluid-guiding elements. For example, just one additional element is arranged between the first wall section and the second wall section. An indirect material joint between the first wall section and the second wall section exists when the first wall section and the second wall section are each joined materially directly to the additional element. However, it is also possible for a plurality of additional elements to be arranged between the first wall section and the second wall section. An indirect material joint between the first wall section and the second wall section exists when the first wall section is materially joined directly to the immediately adjacent additional element, the second wall section is materially joined directly to the immediately adjacent additional element, and the additional elements are materially joined to one another. In the case of an indirect material joint between the two wall sections, provision is preferably made for the direct material joint between the first wall section and the immediately adjacent element, and for the direct material joint between the second wall section and the immediately adjacent element to overlap at least partially in a plan view of the first wall section or the second wall section. In the case of an elongate fluid-guiding element, the connections are preferably extended parallel to one another. As a particular preference, the connections are congruent in the plan view of the first wall section or the second wall section. This applies irrespective of which of the elements listed below forms or form part of the material joint in addition to the wall sections.

According to one preferred embodiment, it is envisaged that at least one supporting layer is arranged in the suction lumen, said layer supporting the connecting tube against collapse, in particular vacuum-induced collapse, and that the first wall section and the second wall section are each materially joined to the supporting layer or one of the supporting layers in order to form the fluid-guiding element or at least one of the fluid-guiding elements. When the connection unit is used as intended, the supporting layer ensures that the connecting tube can withstand the vacuum produced. Including the supporting layer in the material joint between the first wall section and the second wall section ensures that the demands on the contour of the supporting layer are low. To be specific, there must, for example, be no apertures provided in the supporting layer through which the first wall section for the formation of the fluid-guiding element or at least one of the fluid-guiding elements can come into direct contact with the second wall section. If there is only one supporting layer, the first wall section and the second was section are each materially joined directly to the supporting layer. If there is a plurality of supporting layers arranged in a manner stacked one above the other, the wall sections are materially joined directly to the respective supporting layer arranged immediately adjacent, and the supporting layers are materially joined to one another.

According to one preferred embodiment, it is envisaged that the supporting layer is manufactured from polyvinyl chloride, polyurethane, silicone or a mixture thereof. These materials have the advantage that the joining of the wall sections to the supporting layer can be achieved with little effort in terms of manufacturing technology, in particular by means of ultrasonic welding.

According to one preferred embodiment, it is envisaged that the supporting layer is formed by a flat material web section which, in order to support the connecting tube, is formed in a structured manner by raised portions formed integrally with a surface of the flat material web section, wherein a continuous interspace, which allows fluid to pass through in the longitudinal extent of the tube lumen, is formed between the raised portions. A flat material web section designed as above can be obtained at low cost. This results, in particular, from the fact that the design of the raised portions can advantageously be integrated into the production of the flat material web section. A flat material web section is a section of a flat material web. For example, the flat material web section is cut out of a flat material web. The raised portions are preferably already formed in the flat material web. However, it is also possible for the raised portions to be formed only in the flat material web section. In particular, the flat material web is a plastic film web, such that the flat material web section is designed as a plastic film.

The presence of at least one supporting layer is preferred. However, it is also possible to dispense with the presence of the supporting layer. The fluid-guiding structure itself can ensure a certain support for the suction lumen. This support may be sufficient, rendering additional support, e.g. by a supporting layer, unnecessary.

According to one preferred embodiment, it is envisaged that an insert having at least one rib-shaped section is arranged in the suction lumen, and that the first wall section and the second wall section are each materially joined to the rib-shaped section in order to form the fluid-guiding element or one of the fluid-guiding elements. It is also possible to obtain an advantageous fluid-guiding element by connecting the wall sections to the rib-shaped section of the insert. The rib-shaped section has the advantage, in particular, that a reinforcement of the connecting tube can be achieved, this being associated with support of the connecting tube against collapse. In addition, the first wall section and the second wall section are held spaced apart by the rib-shaped section. The result of this is that the cross-sectional area of the suction lumen is reduced less than with direct attachment of the first wall section to the second wall section.

According to one preferred embodiment, it is envisaged that the insert comprises a plurality of interconnected and mutually spaced rib-shaped sections, and that the first wall section and the second wall section are each materially joined to the rib-shaped sections in order to form a plurality of fluid-guiding elements. The formation of a plurality of fluid-guiding elements has the advantage that particularly precise guidance of the fluid flow can be achieved. Because the rib-shaped sections belong to a common insert, the rib-shaped sections can be handled easily together. As a result, manufacture of the connection unit is made easier, e.g. because the positioning of the rib-shaped sections with respect to one another is clearly defined.

According to one preferred embodiment, it is envisaged that the tube wall comprises a first ply and a second ply, which are joined to one another in their edge regions, wherein the first wall section is formed by the first ply, and wherein the second wall section is formed by the second ply. The formation of the tube wall from the two plies makes the connection unit easier to assemble. The first ply and the second ply are preferably designed as film plies. Accordingly, the connecting tube is a film tube. The plies are preferably welded or adhesively bonded to one another in their edge regions.

According to one preferred embodiment, it is envisaged that the tube wall is manufactured from polyvinyl chloride, polyurethane, polyethylene, silicone or a mixture thereof. These materials have the advantage that the first wall section of the tube wall can easily be materially joined to the second wall section, in particular by means of ultrasonic welding.

The tube wall is preferably transparent or translucent. A transparent or translucent tube wall enables visual checking of the suction lumen. For example, it is possible to check whether wound exudate has already entered the suction lumen or how far the wound exudate has already penetrated into the suction lumen. On this basis, it is then possible to decide whether the vacuum wound treatment kit must be replaced or need not be replaced.

The object to be achieved is also achieved by a vacuum wound treatment kit which comprises a vacuum wound dressing and a connection unit having the features described above, wherein the fluid inlet of the connection unit can be fluidically connected to the vacuum wound dressing in a vacuum-tight manner.

In respect of the advantages that can be achieved with the vacuum wound treatment kit, attention is drawn to the relevant statements relating to the connection unit. The features described in connection with the connection unit can be used for the further configuration of the vacuum wound treatment kit.

The object to be achieved is also achieved by a vacuum wound treatment system which comprises a vacuum wound treatment kit having the features described above. The vacuum wound treatment system according to the invention furthermore comprises a vacuum source, wherein the fluid outlet of the connection unit can be fluidically connected to the vacuum source in a vacuum-tight manner.

In respect of the advantages that can be achieved with the vacuum wound treatment system, attention is drawn to the relevant statements relating to the connection unit. The features described in connection with the connection unit can be used for the further configuration of the vacuum wound treatment system.

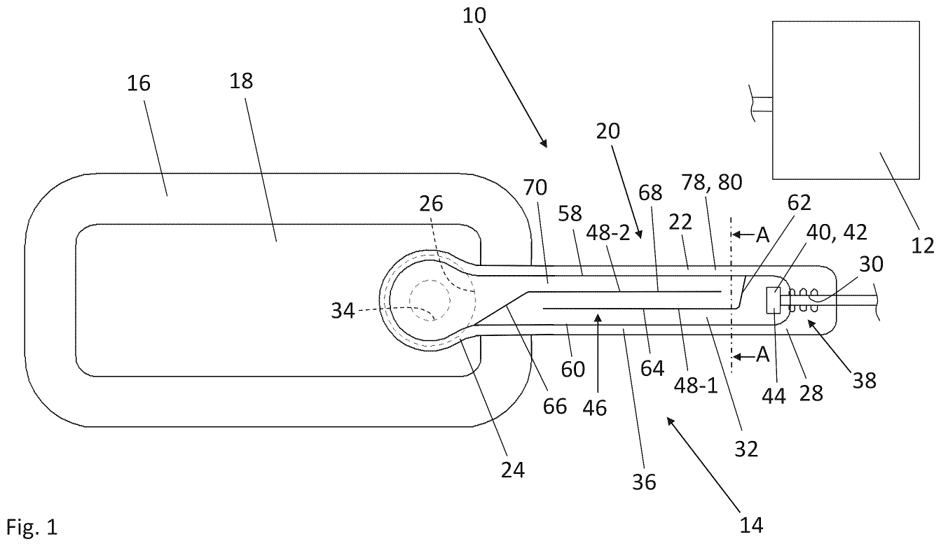

The invention is described below in greater detail by means of the figures, wherein elements that are the same or functionally the same are possibly provided only once with reference signs. The figures serve as an example and should not be interpreted as restrictive. In the figures:
FIG. 1 shows a vacuum wound treatment system comprising a connection unit,

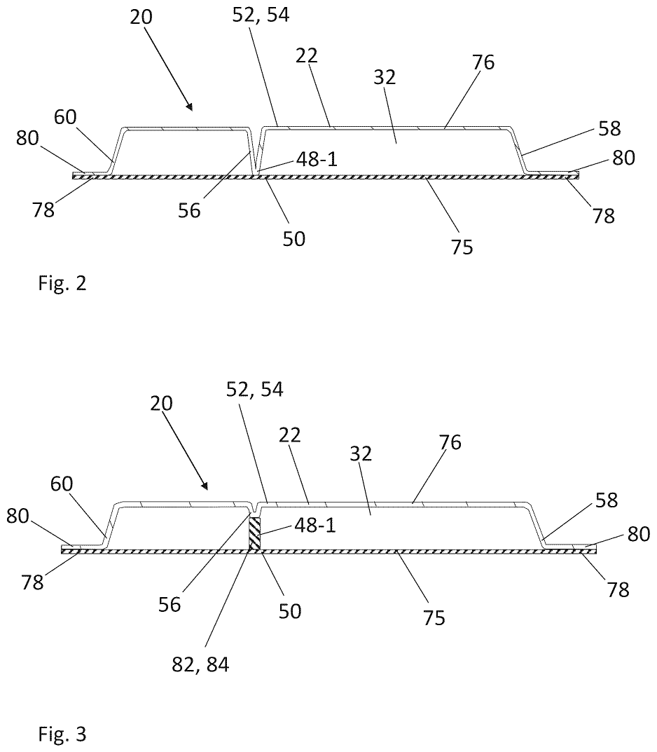

FIG. 2 shows a cross section through a connecting tube of the connection unit shown in FIG. 1,

FIG. 3 shows a cross section through another exemplary embodiment of the connecting tube,

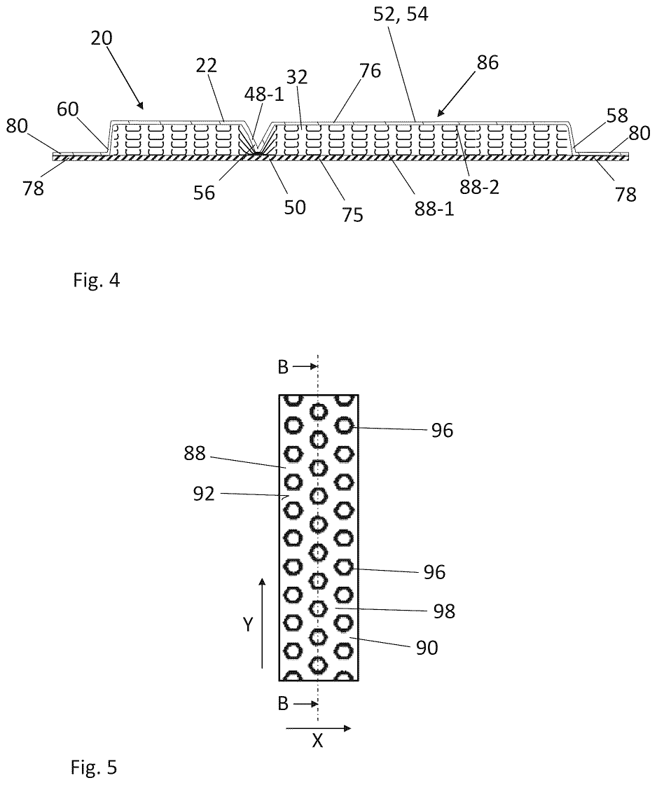

FIG. 4 shows a cross section through another exemplary embodiment of the connecting tube,

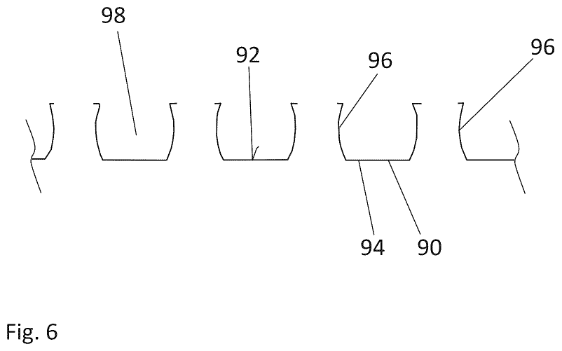

FIG. 5 shows a plan view of a flat material web section, which is arranged as a supporting layer in a suction lumen of the connecting tube shown in FIG. 4,

FIG. 6 shows a section through the flat material web section shown in FIG. 5 along section line B-B, and

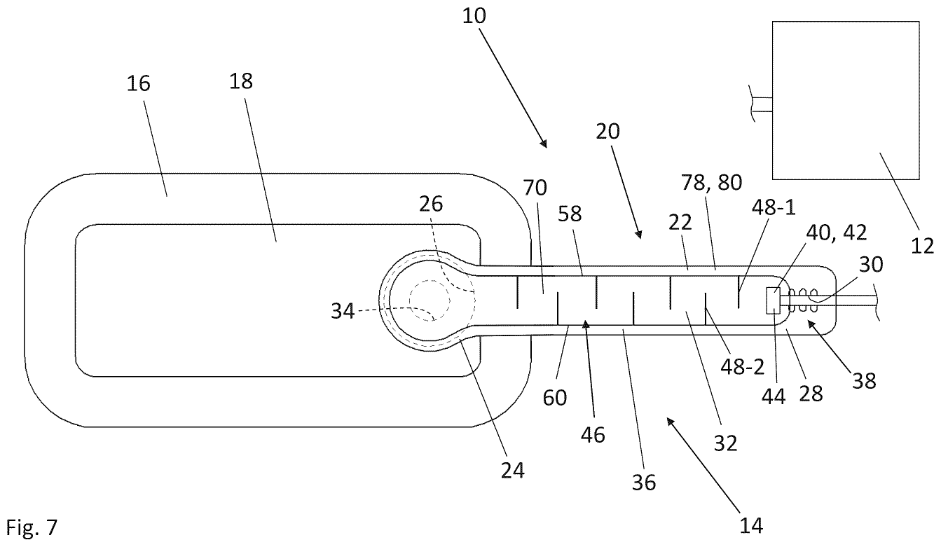

FIG. 7 shows a vacuum wound treatment system comprising a connection unit according to another exemplary embodiment.

FIG. 1 shows a vacuum wound treatment system 10 for use in the vacuum treatment of wounds. The vacuum wound treatment system 10 comprises a vacuum source 12 and a vacuum wound treatment kit 14. The vacuum wound treatment kit 14 comprises a vacuum wound dressing 16, which is referred to below as a wound dressing 16. The wound dressing 16 comprises an air-impermeable covering layer 18 for airtight sealing of a wound. The vacuum wound treatment kit 14 furthermore comprises a connection unit 20. The connection unit 20 comprises an elongate connecting tube 22. The connecting tube 22 comprises a distal end section 24 with a fluid inlet 26 and a proximal end section 28 with a fluid outlet 30. A suction lumen 32 of the connecting tube 22 extends from the fluid inlet 26 to the fluid outlet 30. In the present case, the connecting tube 22 is of single-lumen design.

The fluid inlet 26 can be fluidically connected in a vacuum-tight manner to a connection opening 34 formed in the covering layer 18. In the vacuum wound treatment system 10 illustrated in FIG. 1, the fluid inlet 26 is already connected to the connection opening 34, and therefore the connection opening 34 is covered by the distal end section 24. The distal end section 24 is arranged on the wound dressing 16 in such a way that the connection opening 34 and the fluid inlet 26 of the distal end section 24 are brought at least partially into overlap.

In the exemplary embodiment illustrated, the distal end section 24 is widened over an extended area relative to an elongate central section 36 of the connecting tube 22. In the present case, the distal end section 24 is widened in the form of a circular disk. The widening of the distal end section 24 makes it easier to connect the distal end section 24 to the wound dressing 16.

The fluid outlet 30 of the proximal end section 28 can be fluidically connected to the vacuum source 12 in a vacuum-tight manner. In the case of the exemplary embodiment illustrated, a connection element 38 comprising an inlet 40 and an outlet (not illustrated) is arranged in the fluid outlet 30. An inlet section 42 of the connection element 38, said inlet section comprising the inlet 40, projects through the fluid outlet 30 of the proximal end section 28 into the suction lumen 32. The outlet of the connection element 38 is arranged outside the suction lumen 32 and can be fluidically connected to the vacuum source 12 in a vacuum-tight manner. The fluid outlet 30 of the proximal end section 28 can thus be fluidically connected to the vacuum source 12 in a vacuum-tight manner by means of the connection element 38.

In the exemplary embodiment illustrated, a filter unit 44, which, in the present case, is designed as a PTFE diaphragm filter, is arranged on the inlet section 42 of the connection element 38. The filter unit 44 is assigned to the inlet 40 and is of air-permeable and liquid-impermeable design. According to another exemplary embodiment, the connection element 38 is free from a filter unit.

When the vacuum wound treatment system 10 is used as intended, the wound dressing 16 is applied to a wound. The vacuum source 12 is fluidically connected to the wound dressing 16 in a vacuum-tight manner by the connection unit 20. A vacuum can then be produced in the wound space by the vacuum source 12. The vacuum has the effect that wound exudate is drained from the wound and absorbed by the wound dressing 16, e.g. by an absorption layer of the wound dressing 16. Because the optionally present filter unit 44 is of air-permeable design, the vacuum can be communicated to the wound space through the filter unit 44.

The suction lumen 32 comprises a fluid-guiding structure 46, which is designed to deflect a fluid flow, e.g. a wound exudate flow, flowing through the suction lumen 32 multiple times before it reaches the fluid outlet 30. The design of the fluid-guiding structure 46 is explained in greater detail below with additional reference to FIG. 2. In this regard, FIG. 2 shows a cross section through the connecting tube 22 along the section plane A-A shown in FIG. 1.

The fluid-guiding structure 46 comprises at least one fluid-guiding element 48, which extends in the suction lumen 32. In the exemplary embodiment illustrated in FIGS. 1 and 2, a first fluid-guiding element 48-1 and a second fluid-guiding element 48-2 are present.

The fluid-guiding elements 48 are formed by material joining, in some region or regions, of a first wall section 50 of a tube wall 52 of the connecting tube 22 to a second wall section 54 of the tube wall 52 opposite the first wall section 50. This can be seen in FIG. 2. In the region of the first fluid-guiding element 48-1, the second wall section 54 comprises an indentation 56, which projects in the direction of the first wall section 50.

In the exemplary embodiment illustrated in FIGS. 1 and 2, the first wall section 50 and the second wall section 54 are materially joined directly to one another. Accordingly, no additional element is arranged between the first wall section 50 and the second wall section 54 in the region of the material joint. With regard to the material joining of the first wall section 50 to the second wall section 54, various connection methods may be considered. In the present case, the first wall section 50 and the second wall section 54 are materially joined to one another by welding, in particular by ultrasonic welding, in the region of the fluid-guiding elements 48 to form the fluid-guiding elements 48. The material joints each comprise a weld seam, the profile of which corresponds to the profile of the fluid-guiding element 48 concerned.

According to another exemplary embodiment, the first wall section 50 and the second wall section 54 are joined to one another materially by adhesive bonding, for example.

The suction lumen 32 is delimited laterally by a first side wall 58, extended in the longitudinal extent of the connecting tube 22, and by a second side wall 60, extended in the longitudinal extent of the connecting tube 22. The side walls 58 and 60 are spaced apart and lie opposite one another.

In the present case, the first fluid-guiding element 48-1 has a bent profile. For this purpose, the first fluid-guiding element 48-1 comprises a first section 62 and a second section 64. The first section 62 is arranged adjacent to the proximal end section 28 of the connecting tube 22. Starting from the first side wall 58, the first section 62 extends approximately transversely to the longitudinal extent of the connecting tube 22. The first section 62 ends at a distance from the second side wall 60. The second section 64 adjoins the first section 62 and is extended in the longitudinal extent of the connecting tube 22 in the direction of the distal end section 24.

In the present case, the second fluid-guiding element 48-2 also has a bent profile. For this purpose, the second fluid-guiding element 48-2 comprises a first section 66 and a second section 68. Starting from the second side wall 60, the first section 66 extends obliquely to the longitudinal extent of the connecting tube 22 and ends at a distance from the first side wall 58. The second section 68 adjoins the first section 66 and is extended in the longitudinal extent of the connecting tube 22 in the direction of the proximal end section 28.

The second section 68 of the second fluid-guiding element 48-2 is arranged between the first side wall 58 and the second section 64 of the first fluid-guiding element 48-1. The second section 64 of the first fluid-guiding element 48-1 ends at a distance from the first section 66 of the second fluid-guiding element 48-1. The second section 68 of the second fluid-guiding element 48-2 ends at a distance from the first section 62 of the first fluid-guiding element 48-1.

In consideration of the above-described profile of the fluid-guiding elements 48-1 and 48-2, the fluid-guiding elements 48 define a fluid passage 70 with a meandering course in the suction lumen 32. The fluid passage 70 comprises three longitudinal sections, which extend in the longitudinal extent of the connecting tube 22 and are arranged adjacent to one another. The longitudinal sections are connected to one another by two curved sections.

When the connection unit 20 is used as intended in vacuum treatment, drained wound exudate passes through the fluid inlet 26 into the suction lumen 32. As a fluid flow in the suction lumen 32, the wound exudate first of all flows through a first longitudinal section of the fluid passage 70 in the direction of the proximal end section 28. The wound exudate is then deflected by the first section 62 of the first fluid-guiding element 48-1 and enters an adjoining second longitudinal section of the fluid passage 70. In the second longitudinal section, the flow direction of the wound exudate is opposite to the flow direction in the first longitudinal section. Accordingly, the wound exudate in the second line section flows back in the direction of the distal end section 24. When it reaches the first section 66 of the second fluid-guiding element 48-2, the fluid flow is deflected again by the first section 66 and then flows through a third of the longitudinal sections to the proximal end section 28 and the fluid outlet 30.

The multiple deflection of the fluid flow in the suction lumen 32 prevents the fluid flow from reaching the proximal end section 28 and the fluid outlet 30 in a surge or surges. Instead, a fluid flow surging in is broken by the fluid-guiding structure 46, such that the wound exudate only reaches the proximal end section 28 when the absorption capacity of the wound dressing 16 has been exceeded and, as a consequence, the suction lumen 32 is filled continuously with wound exudate.

If the fluid flow were to reach the proximal end section 28 in a surge or surges, the filter element 44 could be prematurely clogged by wound exudate. The wound dressing 16 would then have to be changed before the absorption capacity of the wound dressing 16 had been reached. As mentioned above, the filter element 44 is optional. If the filter element 44 were not present, the fluid flow reaching the proximal end section 28 in a surge or surges could get into the downstream vacuum source 12. This could contaminate and/or damage the vacuum source 12.

In the exemplary embodiment illustrated in the figures, the tube wall 52 comprises a lower or first ply 75 and an upper or second ply 76. An edge region 78 of the first ply 75 is joined to an edge region 80 of the second ply 76. The plies 75 and 76 jointly surround the suction lumen 32.

In the present case, the plies 75 and 76 are designed as film plies. Accordingly, the connecting tube 22 is a film tube. The plies 75 and 76 are preferably manufactured from polyurethane, polyvinyl chloride, polyethylene, silicone or a mixture thereof. The edge regions 78 and 80 are preferably joined to one another by a welded joint. In the exemplary embodiment illustrated, the fluid inlet 26 of the distal end section 24 is formed in the first ply 75. The fluid outlet 30 is formed between the edge region 78 of the first ply 75 and the edge region 80 of the second ply 76.

The tube wall 52 is preferably of transparent or translucent design. Visual checking of the suction lumen 32 is thereby made possible. For example, it is possible to check whether wound exudate has already entered the suction lumen 32 or how far the wound exudate has already penetrated into the suction lumen 32. On this basis, it is then possible to decide whether the vacuum wound treatment kit 14 must be replaced or need not be replaced. Here, the lengthening of the flow path associated with a meandering fluid passage 70 enables particularly accurate adaptation of a suitable time for the replacement of the vacuum wound treatment kit 14.

FIG. 3 shows a cross section, corresponding to FIG. 2, through another exemplary embodiment of the connecting tube 22. Here, the assumption is that the profile of the fluid-guiding elements 48 corresponds to the profile illustrated in FIG. 1.

In the exemplary embodiment illustrated in FIG. 3, an insert 82 with a rib-shaped section 84 is arranged in the tube lumen 32. To form the first fluid-guiding element 48-1, the wall sections 50 and 54 are each materially joined directly to the rib-shaped section 84, preferably by means of a welded joint. The insert 82 preferably comprises a further rib-shaped section (not illustrated), wherein the wall sections 50 and 54 are each materially joined directly to the further rib-shaped section to form the second fluid-guiding element 48-2. In the exemplary embodiment illustrated in FIG. 3, the wall sections 50 and 54 are therefore materially joined indirectly to one another, namely by means of the rib-shaped section 84.

FIG. 4 shows a cross section, corresponding to FIG. 2, through another exemplary embodiment of the connecting tube 22. Here, the assumption is that the profile of the fluid-guiding elements 48 corresponds to the profile illustrated in FIG. 1.

In the exemplary embodiment illustrated in FIG. 4, a supporting unit 86 is arranged in the suction lumen 32, said unit supporting the connecting tube 22 against collapse, in particular vacuum-induced collapse, and allowing fluid to pass through in the longitudinal extent of the suction lumen 32. The supporting unit 86 comprises a plurality of supporting layers 88, which are arranged in such a way as to be stacked one above the other in the suction lumen 32. In the present case, four supporting layers 88 are present. However, there may also be a different number of supporting layers 88.

To form the first fluid-guiding element 48-1, the first wall section 50 is joined materially in some region or regions to an immediately adjacent first supporting layer 88-1. The second wall section 54 is joined materially in some region or regions to an immediately adjacent second supporting layer 88-2. Moreover, the supporting layers 88 are materially joined to one another. In the exemplary embodiment illustrated in FIG. 4 too, therefore, the wall sections 50 and 54 are materially joined indirectly to one another, namely by means of the supporting layers 88.

The design of the supporting layers 88 is explained in greater detail below by means of a preferred exemplary embodiment with additional reference to FIGS. 5 and 6. In this regard, FIG. 5 shows a plan view of one of the supporting layers 88. It should be noted here that FIG. 5 illustrates only a segment of the supporting layer 88. Accordingly, the actual outer contour of the supporting layer 88 differs from the outer contour shown in FIG. 5. FIG. 6 shows a section through the supporting layer 88 along the section line B-B shown in FIG. 5.

The supporting layer 88 is formed by a flat material web section 90. The flat material web section 90 is extended in two surface directions X and Y. In order to support the connecting tube 22, a first side 92 of the flat material web section 90 is formed in a structured manner by raised portions 96 formed integrally with a surface 94 of the flat material web section 90. A continuous interspace 98 is formed between the raised portions 96, thus enabling fluid, such as wound exudate, to be carried through the interspace 98.

In the present case, the flat material web section 90 is a plastic film 90. The plastic film 90 is preferably manufactured from polyethylene, polyurethane, polyvinyl chloride or a mixture thereof. These plastics have the advantage that the above-described indirect material joining between the first wall section 50 and the second wall section 54 can be achieved in a single method step. The indirect material joining can preferably be achieved by welding. In this case, the first wall section 50, the second wall section 54 and the interposed supporting layers 88 are heated in certain regions to a temperature above their melting temperature and are thereby welded together.

The abovementioned plastics furthermore have the advantage that a flexible connecting tube 22 is obtained, this being associated with high patient comfort. Thus, the flexible embodiment of the connecting tube 22 makes it possible to avoid the occurrence of pressure ulcers, for example. Moreover, the abovementioned plastics also have adequate rigidity. Thus, the plastics ensure that the continuous interspace 98 is maintained during the use of the connection unit 22 despite the vacuum produced.

FIG. 7 shows the vacuum wound treatment system 10 comprising a connection unit 20 according to another exemplary embodiment. The connection unit illustrated in FIG. 7 differs from the connection unit 20 illustrated in FIG. 1 in respect of the number and arrangement of the fluid-guiding elements 48.

In the exemplary embodiment illustrated in FIG. 7, a multiplicity of fluid-guiding elements 48 is present, which extend in the suction lumen 32 transversely to the longitudinal extent of the connecting tube 22. Starting from the first side wall 58, a group of first fluid-guiding elements 48-1 extends beyond the longitudinal center line of the connecting tube 22 and ends at a distance from the second side wall 60. Starting from the second side wall 60, a group of second fluid-guiding elements 48-2 extends beyond the longitudinal center line of the connecting tube 22 and ends at a distance from the first side wall 58. In the present case, the fluid-guiding elements 48 are arranged in a manner distributed such that, when viewed in the longitudinal extent of the connecting tube 22, a first fluid-guiding element 48-1 is always followed by a second fluid-guiding element 48-2. Accordingly, a second fluid-guiding element 48-2 is always followed by a first fluid-guiding element 48-1.

In view of the above-described arrangement and design of the fluid-guiding elements 48, the fluid-guiding structure 46 also defines a meandering fluid passage 70 in the suction lumen 32 in the exemplary embodiment illustrated in FIG. 7. As it passes through the fluid passage 70, the wound exudate is deflected multiple times, and therefore the same advantageous effect is achieved that was previously explained in connection with the exemplary embodiment illustrated in FIGS. 1 and 2.

In the exemplary embodiment illustrated in FIG. 7, the first wall section 50 and the second wall section 54 are materially joined directly to one another to form the fluid-guiding elements 48. Alternatively, the fluid-guiding elements 48 are formed by indirect material joining of the first wall section 50 to the second wall section 54, as described above in connection with FIGS. 3 and 4.

1-20. (canceled)

21. A connection unit for vacuum-tight fluidic connection of a vacuum wound dressing to a vacuum source, comprising:
an elongate single-or multi-lumen connecting tube, having a distal end section with a fluid inlet and a proximal end section with a fluid outlet, wherein the fluid inlet is able to be fluidically connected to the vacuum wound dressing in a vacuum-tight manner,
wherein the fluid outlet is able to be fluidically connected to the vacuum source in a vacuum-tight manner, and wherein a suction lumen of the connecting tube extends from the fluid inlet to the fluid outlet,
wherein the suction lumen comprises a fluid-guiding structure, which is able to deflect a fluid flow flowing through the suction lumen multiple times before it reaches the fluid outlet, wherein to deflect the fluid flow, the fluid-guiding structure comprises at least one fluid-guiding element extending in the suction lumen, and
wherein the fluid-guiding element is formed by material joining, in some region or regions, a first wall section of a tube wall of the connecting tube to a second wall section of the tube wall opposite the first wall section.

22. The connection unit of claim 21, wherein the material joint between the first wall section and the second wall section is in the form of a seam.

23. The connection unit of claim 21, wherein the fluid-guiding structure comprises at least two fluid-guiding elements.

24. The connection unit of claim 21, wherein the fluid-guiding structure is able to block any straight-line connection between an imaginary first plane, which is arranged in the distal end section and is aligned orthogonally with respect to the longitudinal extent of the connecting tube, and an imaginary second plane, which is arranged in the proximal end section and is aligned orthogonally with respect to the longitudinal extent of the connecting tube.

25. The connection unit of claim 21, wherein the fluid-guiding structure is able to deflect the fluid flow at least once through an angle which is at least 90.

26. The connection unit of claim 21, wherein the flow direction of the fluid flow in a first section of the suction lumen is opposite to the flow direction of the fluid flow in a second section of the suction lumen.

27. The connection unit of claim 21, wherein the fluid-guiding structure defines a meandering or labyrinthine fluid passage in the suction lumen.

28. The connection unit of claim 21, wherein the fluid-guiding element or at least one of the fluid-guiding elements extends transversely to the longitudinal extent of the connecting tube, at least in some section or sections.

29. The connection unit of claim 21, wherein the fluid-guiding element or at least one of the fluid-guiding elements extends in the longitudinal extent of the connecting tube, at least in some section or sections.

30. The connection unit of claim 21, wherein the fluid-guiding element or at least one of the fluid-guiding elements is formed by direct material joining of the first wall section to the second wall section.

31. The connection unit of claim 21, wherein the fluid-guiding element or at least one of the fluid-guiding elements is formed by indirect material joining of the first wall section to the second wall section.

32. The connection unit of claim 31, wherein at least one supporting layer is arranged in the suction lumen, said layer supporting the connecting tube against collapse, and wherein the first wall section and the second wall section are each materially joined to the supporting layer or one of the supporting layers in order to form the fluid-guiding element or at least one of the fluid-guiding elements.

33. The connection unit of claim 32, wherein the supporting layer is manufactured from polyvinyl chloride, polyurethane, polyethylene, silicone or a mixture thereof.

34. The connection unit of claim 31, wherein an insert having at least one rib-shaped section is arranged in the suction lumen, and in that the first wall section and the second wall section are each materially joined to the rib-shaped section in order to form the fluid-guiding element or one of the fluid-guiding elements.

35. The connection unit of claim 34, wherein the insert comprises a plurality of interconnected and mutually spaced rib-shaped sections, and wherein the first wall section and the second wall section are each materially joined to the rib-shaped sections in order to form a plurality of fluid-guiding elements.

36. The connection unit of claim 21, wherein the tube wall comprises a first ply and a second ply, which are joined to one another in their edge regions, wherein the first wall section is formed by the first ply, and wherein the second wall section is formed by the second ply.

37. The connection unit of claim 21, wherein the tube wall is manufactured from polyvinyl chloride, polyurethane, polyethylene, silicone or a mixture thereof.

38. The connection unit of claim 21, wherein the tube wall is transparent or translucent.

39. A vacuum wound treatment kit comprising a vacuum wound dressing and a connection unit as claimed in claim 21, wherein the fluid inlet of the connection unit is able to be fluidically connected to the vacuum wound dressing in a vacuum-tight manner.

40. A vacuum wound treatment system comprising a vacuum wound treatment kit as claimed in claim 39, and comprising a vacuum source, wherein the fluid outlet of the connection unit is able to be fluidically connected to the vacuum source in a vacuum-tight manner.
